(19) United States
(12) Patent Application Publication
(10) Pub. No.: US 2009/0209743 A1
(43) Pub. Date: Aug. 20, 2009

(54) COLOURED ORGANOPOLYSILOXANES

(76) Inventors: Roxana Barbieru, Frankfurt (DE); Stefan Ehrenberg, Hattersheim (DE); Werner Russ, Flörsheim-Wicker (DE); Christian Ochs, Burghausen (DE); Daniel Schildbach, Altötting (DE)

Correspondence Address: CONNOLLY BOVE LODGE & HUTZ, LLP, P O BOX 2207, WILMINGTON, DE, 19899, US

(21) Appl. No.: 12/304,841

(22) PCT Filed: Jun. 6, 2007
(86) PCT No.: PCT/EP2007/055589

§ 371 Date: Feb. 12, 2009

(30) Foreign Application Priority Data
Jun. 14, 2006 (DE) 10 2006 027 533.0

(51) Int. Cl. C09B 29/36; C07D 251/52

(52) U.S. Cl. 534/726; 544/208

(57) ABSTRACT

The present invention relates to colored organopolysiloxanes comprising units of the formula

R¹ₐ(RO)_{b}A_{c}R²_{d}SiO_{(4-a-b-c-d)/2} (I),

in which
R can be identical or different and is hydrogen or a monovalent, unsubstituted or substituted hydrocarbon radical;
R¹ can be identical or different and is hydrogen or a monovalent, SiC-bonded, unsubstituted or substituted hydrocarbon radical;
R² can be identical or different and is a substituted monovalent hydrocarbon radical;
A can be identical or different and is an organic dye radical free from sulfonic acid groups and sulfonate groups;
a is 0, 1, 2 or 3;
b is 0, 1, 2 or 3;
d is 0, 1, 2 or 3;
and c is 0, 1 or 2;
   with the proviso that the sum a+b+c+d is ≦3, the organopolysiloxane has at least one radical A per molecule, and in the units of the formula (I) where c is other than 0 when d is 0. The invention also relates to a process to the color organopolysiloxane.

The present invention relates to functionalized silicone compounds to which chromophoreic molecules are covalently attached, to processes for preparing them, and to the use of these colored silicone compounds.

The simultaneous use of silicon compounds and dyes is problematic owing to the immiscibility or insolubility of the majority of dyes in silicon compounds. The lack of compatibility between the two classes of substance therefore often leads to inhomogeneously colored products and/or to slow exudation (migration) of one of the product's components, and hence to product properties that are negative overall. The use of defined physical blends of dyes with specific silanes and/or siloxanes, as described for example in U.S. Pat. No. 5,281,240, may counter these adverse consequences to a certain degree, but cannot be used for long-lasting prevention of separation of the individual components.

The problem can be solved, in contrast, if the dye molecule is bonded chemically to an organosilicon compound.

Thus it is the case that silanes with a dye content have been known for a number of decades. They are a topic of numerous monographs and patents (in this regard see, for instance, J. Soc. Dyers and Col. 1969, 85 (9), pp. 401-404).

Dye-carrying silanes are described for the first time by U.S. Pat. No. 2,925,313. In that case the conventional synthesis of azo dyes via azo coupling is modified by employing aniline-modified silanes as a coupling component. According to GB 2018804, phenyl-containing silanes are also suitable for this purpose. The silane-containing dyes obtained in this way are subsequently polymerized to give the corresponding polysiloxanes.

EP 0336709 A2 discloses organopolysiloxanes having triazine-containing radicals, which act as optical brighteners for synthetic fibers and paper. In this instance the bond is forged through the reaction of a sulfonic acid group of the optical brightener with an amino-functional silane or siloxane, to give the sulfonamide. Silicone compounds with nitroaromatic dye radicals can be obtained, according to U.S. Pat. No. 4,403,099, by reacting epoxy-functional siloxanes under basic conditions with amine- or sulfonamide-containing nitro dyes. As an alternative to this, U.S. Pat. No. 4,405,801 proposes bonding ring-halogenated aromatic nitro dyes to amino-functional siloxanes by means of nucleophilic substitution on the aromatic ring.

U.S. Pat. No. 6,918,931 describes yellow dye-carrying siloxane prepolymers for producing intraocular lenses. The covalent attachment of the chromophore takes place in this case either via diisocyanate coupling of siloxanes which carry carbinol groups and of reactive dyes which likewise carry carbinol groups, or via a platinum-catalyzed hydrosilylation reaction between an SiH-modified reactive dye and a terminal divinylpolysiloxane. Both processes are not accomplished without substantial volumes of solvents, the first version employing diisocyanates, which are difficult to handle from a toxicological standpoint, while the second version employs platinum catalysts, which are expensive. Expensive transition metal catalysts comprising platinum or rhodium are likewise employed by US 2005/0, 100,945.

A feature common to all of the abovementioned preparation processes is that they are restricted either only to selected dyes or dye precursors, such as aniline-containing azo compounds, amine-, sulfonic acid- or sulfonamide-containing chromophores, and unhalogenated or halogenated nitroaromatics, for example, or exclusively to specific silicone oils. Moreover, on account of the preparation processes employed, which require highly specific and in some cases highly drastic or laborious reaction conditions, the siloxanes disclosed in the cited patent literature also do not contain any further functional groups. Additional disadvantages, furthermore, are the use of toxicologically objectionable chromophores based on aniline or nitroaromatics, the reaction yields, which are often very low, and the relatively complicated syntheses over two or more reaction steps.

WO 98/40429 A1 has already described the preparation of organopolysiloxanes comprising dye radicals through the reaction of nucleophilic polysiloxanes with water-soluble reactive dyes containing sulfonic acid groups and/or sulfonate groups. A disadvantage of this synthesis process, however, is the use of polar, water-soluble reactive dyes which are therefore highly olophobic, with the consequent need either for a heterogeneous reaction regime, in which case a quantitative yield is achievable only with great difficulty, or the use of relatively large volumes of compatibilizing solvents, which must be removed again, at cost and inconvenience, following the preparation. Furthermore, the introduction of polar dye radicals into the siloxane chain leads to a reduction in the hydrophobic properties of the overall polymer. Although in certain applications this may be advantageous, it is nevertheless undesirable in the majority of cases, as with the hydrophobization of leather, the coloring of silicone elastomers, and similar applications.

The present invention provides colored organopolysiloxanes comprising units of the formula

R¹ₐ(RO)_{b}A_{c}R²_{d}SiO_{(4-a-b-c-d)/2} (I),

in which
R can be identical or different and is hydrogen or a monovalent, unsubstituted or substituted hydrocarbon radical;
R¹ can be identical or different and is hydrogen or a monovalent, SiC-bonded, unsubstituted or substituted hydrocarbon radical;
R² can be identical or different and is a substituted monovalent hydrocarbon radical;
A can be identical or different and is an organic dye radical free from sulfonic acid groups and sulfonate groups;
a is 0, 1, 2 or 3;
b is 0, 1, 2 or 3;
d is 0, 1, 2 or 3; and
c is 0, 1 or 2;
with the proviso that the sum a+b+c+d is ≦3, the organopolysiloxanes have, at least one radical A per molecule, and in the units of the formula (I) where c is other than 0 d is 0.

In the context of the present invention, the term “organopolysiloxanes” embraces not only polymeric but also oligomeric and dimeric siloxanes.

R is preferably hydrogen or a hydrocarbon radical having 1 to 18, in particular 1 to 8, carbon atoms, which may be substituted and/or interrupted by one or more oxygen atoms.

Examples of R are (C₁-C₁₈)-alkyl radicals, such as methyl, ethyl, n-propyl, isopropyl, n-butyl, sec-butyl, isobutyl, tert-butyl, n-pentyl, isopentyl, neopentyl, tert-pentyl radical, hexyl, particularly n-hexyl, heptyl, particularly n-heptyl, octyl, particularly n-octyl and isooctyl, such as 2,2,4-trimethylpentyl, nonyl, particularly n-nonyl, decyl radicals, particularly n-decyl, dodecyl, particularly n-dodecyl, and octadecyl, particularly n-octadecyl; (C₃-C₁₀)-cycloalkyl radicals, such as cyclopentyl, cyclohexyl, cycloheptyl, and methylcyclohexyl; (C₂-C₆)-alkenyl radicals, such as vinyl, allyl (2-propenyl) and isoallyl (1-propenyl); aryl radicals, such as phenyl, naphthyl, anthryl, and phenanthryl;

(C₁-C₄)-alkylaryl radicals, such as o-, m-, and p-tolyl, xylyl, and ethylphenyl; and aryl-(C₁-C₄)-alkyl radicals, such as benzyl and α- and β-phenylethyl radicals.

More preferably R is hydrogen, methyl, ethyl or propyl.

R¹ is preferably hydrogen or a hydrocarbon radical having 1 to 18, in particular 1 to 8 carbon atoms, which may be substituted and/or interrupted by one or more oxygen atoms.

Examples of R¹ are the radicals specified for R, and additionally haloalkyl radicals, such as 3,3,3-trifluoro-n-propyl, 2,2,2,2′,2′,2′-hexafluoroisopropyl, heptafluoroisopropyl, and also haloaryl radicals, such as o-, m-, and p-chlorophenyl, for example.

More preferably R¹ is methyl, vinyl or allyl.

R² is preferably a substituted hydrocarbon radical having 1 to 200 carbon atoms, which may be interrupted by one or more heteroatoms, such as oxygen, sulfur or nitrogen.

R² is more preferably a hydrocarbon radical which carries amino, hydroxyl, mercapto, epoxy or a carboxylic acid substituent or derivatives thereof and which may be interrupted, furthermore, by one or more nitrogen, oxygen or sulfur atoms.

With particular preference R² is a hydrocarbon radical having 1 to 20 carbon atoms which is substituted by amino, hydroxyl, mercapto, epoxy or carboxylic acid substituents or derivatives thereof.

Examples of R² are

a) hydrocarbon radicals substituted by amino groups and derivatives thereof, such as aminomethyl, phenylaminomethyl, aminopropyl, aminoethylaminopropyl, cyclohexylaminopropyl and acylated aminopropyl, for example;
b) hydrocarbon radicals substituted by hydroxyl groups, such as primary, secondary or tertiary alcohol radicals, such as 3-hydroxypropyl and 4-hydroxybutyl, or hydrocarbon radicals which carry aromatic hydroxyl groups, such as the phenol or eugenol radical, for example;
c) hydrocarbon radicals substituted by mercapto groups, such as 3-mercaptopropyl, for example;
d) hydrocarbon radicals substituted by epoxy groups, such as those, for example, from the group consisting of

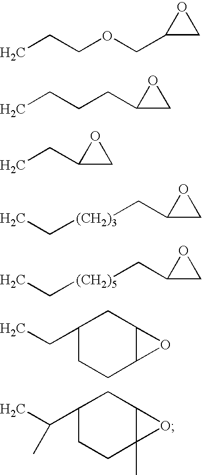

e) hydrocarbon radicals substituted by carboxylic acid groups or derivatives thereof, such as, for example, alkanoic acid radicals, such as the acetyl, 3-carboxypropyl, 4-carboxybutyl, 10-carboxydecyl, and 3-(ethane-1,2-dicarboxyl) propyl radical, acid anhydride radicals, such as the 3-(2,5-dioxotetrahydrofuranyl)propyl radical, and ester radicals, such as the undecene silyl ester radical;
f) hydrocarbon radicals substituted by carbonyl groups, such as ketone-functional radicals and aldehyde-functional radicals, such as the propionaldehyde radical, for example;
g) hydrocarbon radicals substituted by acrylate or methacrylate groups, such as 3-acryloyloxypropyl and 3-methacryloyloxypropyl, for example;
h) SiC- or SiOC-bonded hydrocarbon radicals substituted by polyether groups, such as those derived from polyethylene glycol, polypropylene glycol, poly-(1,4-butanediol) and copolymers thereof, such as the propylpolyglycol radical, for example;
i) hydrocarbon radicals substituted by quaternary nitrogen atoms, such as —(CH₂)₃—N(CH₃)₃+X) and —(CH₂)₃—NH—CH₂—CH(OH)—CH₂—N(CH₃)₂Cl₂H₂₅⁺X⁻, for example, X⁻ being a suitable anion;
j) hydrocarbon radicals substituted by phosphonato groups, such as phosphonatoalkyl radicals, for example;
k) hydrocarbon radicals substituted by silalactone groups;
l) hydrocarbon radicals substituted by glycoside groups, such as those, for example, in which the glycoside radical, which may be composed of 1 to 10 monosaccharide units, is attached via an alkylene or oxyalkylene spacer.

R² is more preferably aminopropyl, aminoethylaminopropyl, hydroxypropyl or mercaptopropyl.

A dye radical represented by A is preferably the radical of an azo, anthraquinone, oxyquinophthalone, coumarin, naphthalimide, benzoquinone, naphthoquinone, flavone, anthrapyridone, quinacridone, xanthene, thioxanthene, benzoxanthene, benzothioxanthene, perylene, perinone, acridone, phthalocyanine, methine, diketopyrrolopyrrole, triphendioxazine, phenoxazine, or phenothiazine dye or of a metal complex compound thereof.

The dye radical A may be bonded to the unit of the formula (I) via a bond, i.e., as a monovalent radical, or else as a polyvalent radical. In the latter case, therefore, dye

A joins two or more sil(oxan)yl radicals to one another.

Examples of dye radicals A are in particular the radicals A1 to A38 below.

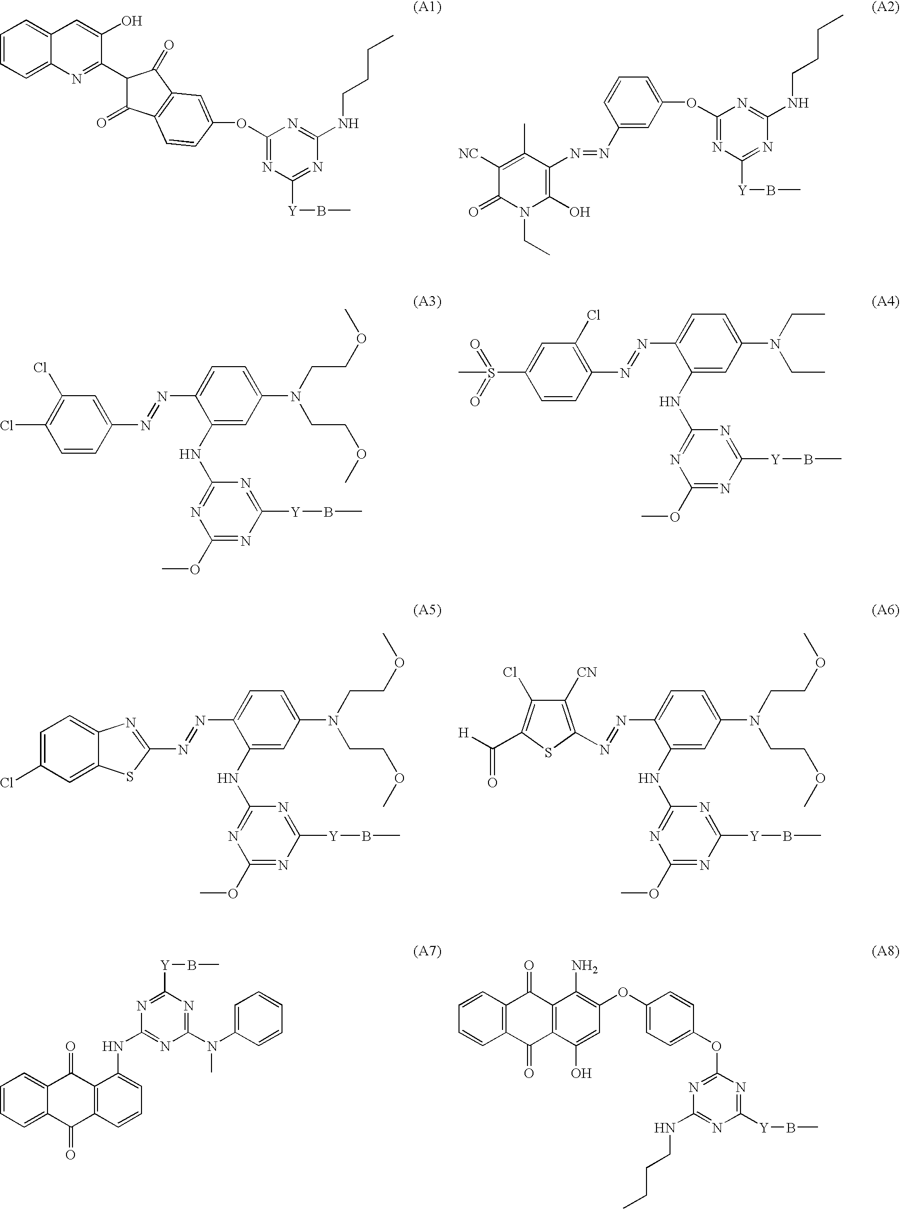

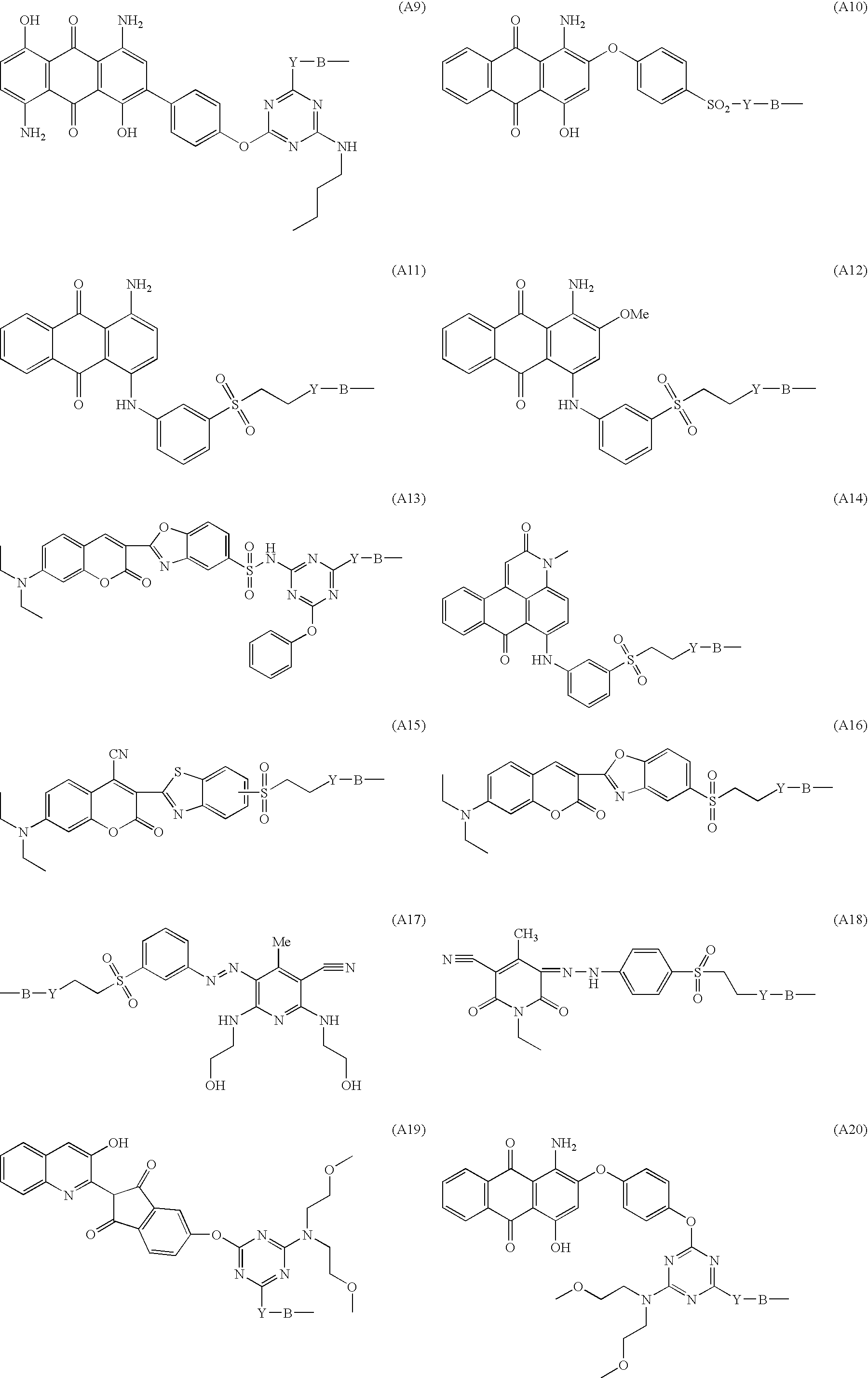

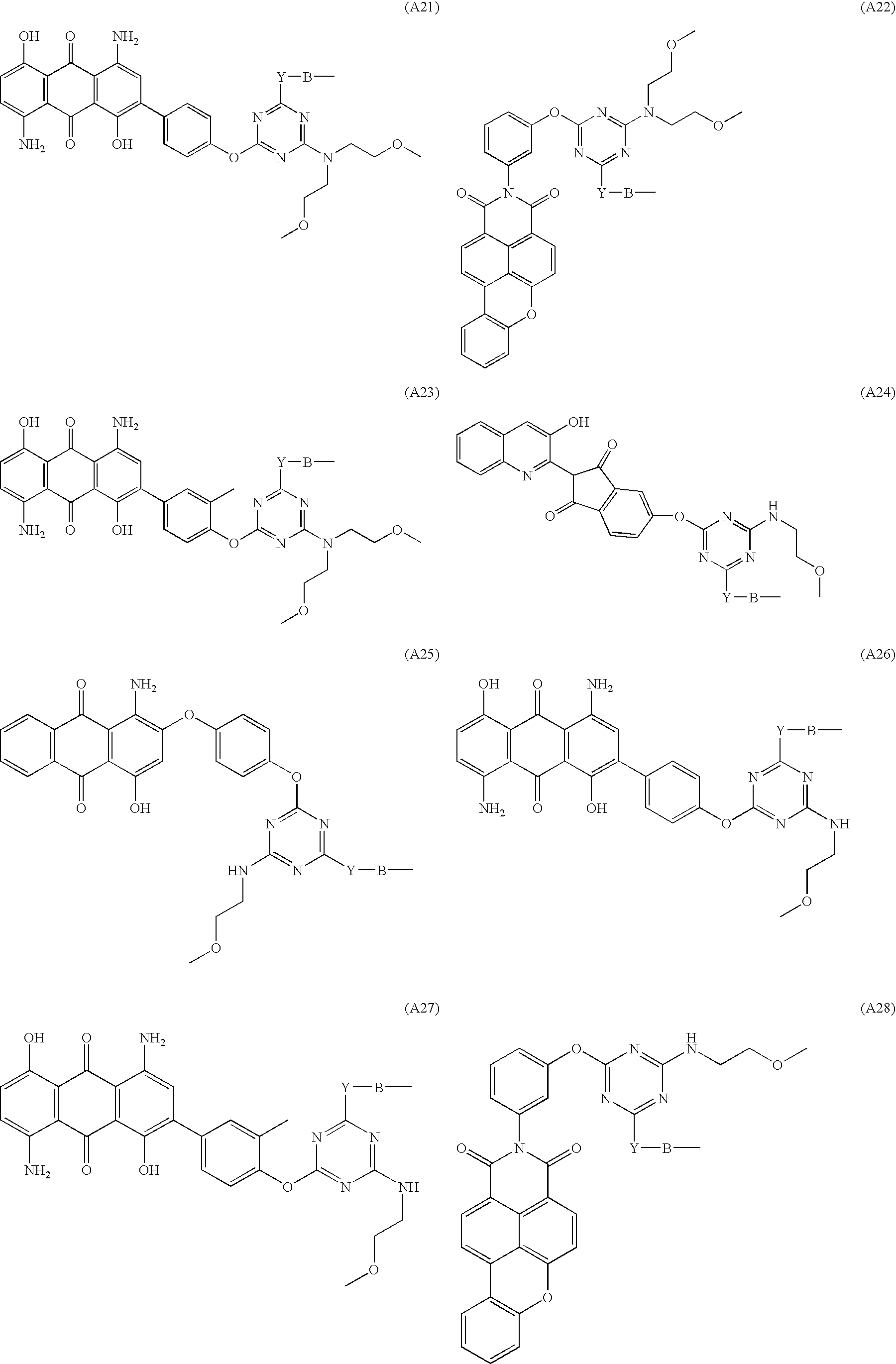

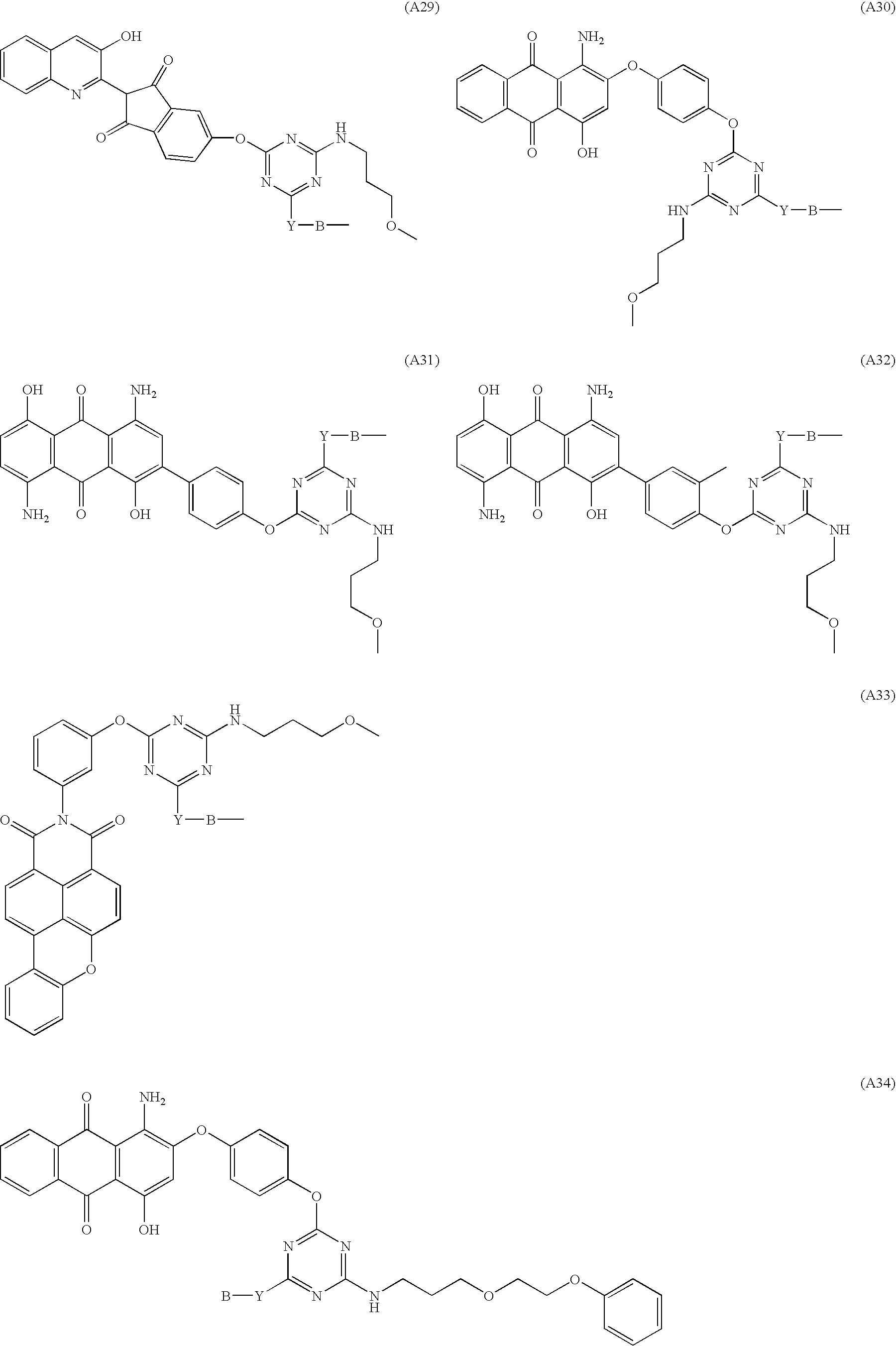

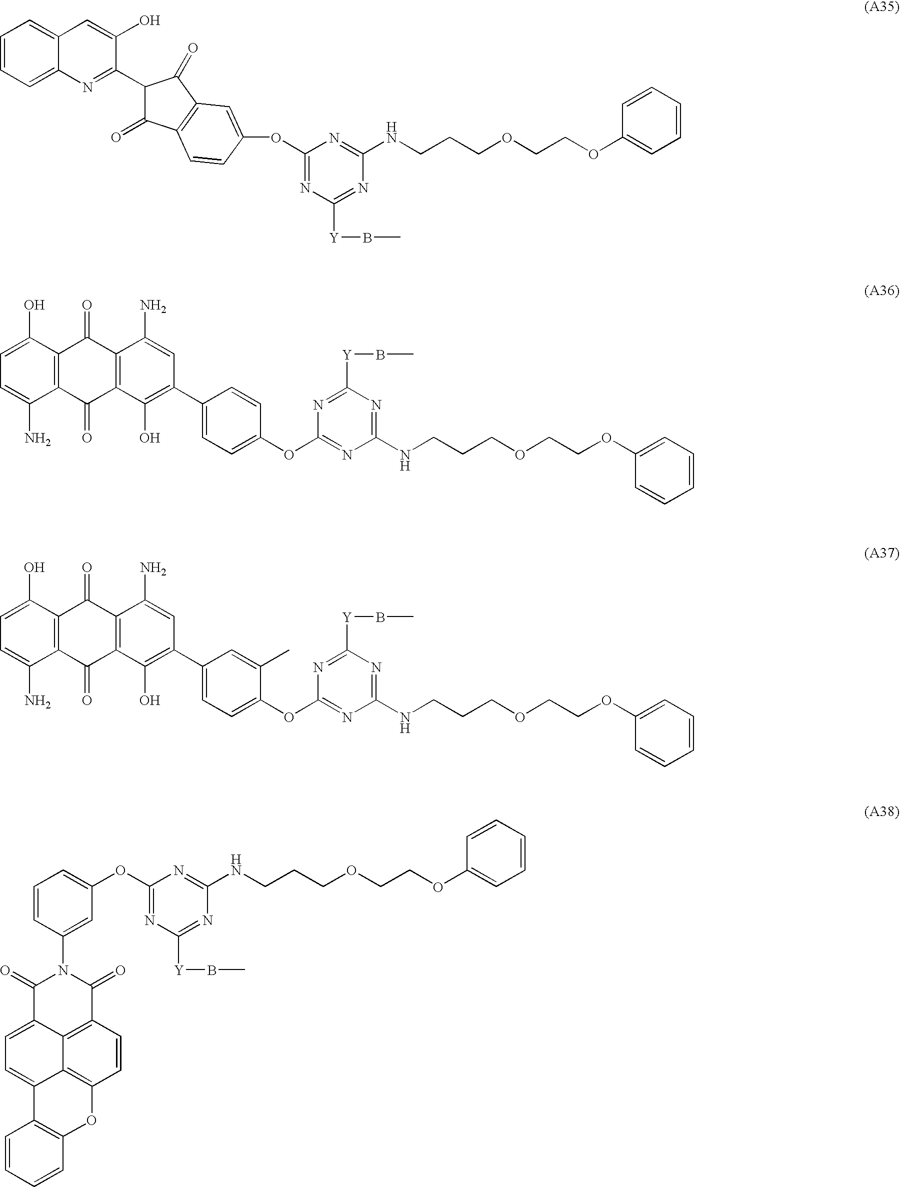

in which
Y is —O—, —S— or —NR³— and R³ is hydrogen or (C₁-C₄)-alkyl;
and B is a divalent bridge.

B connects the dye chromophore to a silicon atom of the silicon and is preferably a hydrocarbon radical, which may be unsubstituted or substituted and/or interrupted by one or more heteroatoms, such as oxygen, nitrogen, and sulfur.

B is preferably a divalent linear (C₁-C₃₀)-hydrocarbon radical unsubstituted or substituted and/or interrupted by one or more heteroatoms, such as oxygen, nitrogen, and sulfur. Particular preference is given to unsubstituted or substituted (C₁-C₁₀)-alkylene radicals, such as methylene, ethylene, propylene, butylene, 4-azahexylene, 1-hydroxyethylene, 4-oxa-6-hydroxyheptylene, for example, and also alkylene groups substituted by a maximum of 4 sugar radicals.

In the units of the formula (I) c is preferably 0 or 1 and d is likewise 0 or 1, with d being 0 if c is 1.

Preferred organopolysiloxanes of the invention are those in which in at least 50%, more preferably in at least 80%, and very preferably in at least 90% of all of the units of the formula (I) the sum of a+b+c+d is 2.

Particularly preferred organopolysiloxanes of the invention are of the formula (II)

AₘR²ₙR¹₃₋ₘ₋ₙSiO-(A₂SiO)ₑ—(R¹_{f}R²_{2-f}SiO)_{g}—(R²ₕR¹₂₋ₕSiO)ᵢ—(R¹ⱼAR²₁₋ⱼSiO)ₖ—SiAₘR²ₙR¹₃₋ₘ₋ₙ (II)

in which
R¹, R² and A are defined as specified above;
f is 0 or 1, preferably 1;
h is 0, 1 or 2, preferably 0;
j is 0 or 1, preferably 1;
m is 0 or 1;
n is 0 or 1;
e is 0 or an integer from 1 to 100;
g is 0 or an integer from 1 to 100;
i is 0 or an integer from 1 to 100;
k is an integer from 1 to 100;
it being possible for the subunits in the formula (II) to be distributed randomly in the molecule.

The viscosities of the organopolysiloxanes of the invention range from preferably 1 mm²/s through to a consistency at which they are solid or waxlike at room temperature. Particular preference is given to the viscosity range between 10 mm²/s and 10 000 000 mm²/s. Particular preference is given to the viscosity range between 100 mm²/s and 500 000 mm²/s, and also the range of solid or waxlike consistency at room temperature.

The dye content of the organopolysiloxanes of the invention is preferably 0.1% to 90% by weight, more preferably 0.5% to 50% by weight, in particular 5% to 25% by weight, based in each case on the total weight of the organopolysiloxane of the invention.

Examples of the organopolysiloxanes of the invention are the compounds below of the formulae (IIa) to (IId)

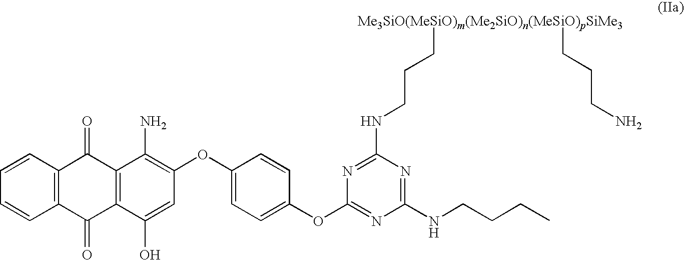

in which Me is methyl and m is 1, n is 55, and p is 2 to 3;

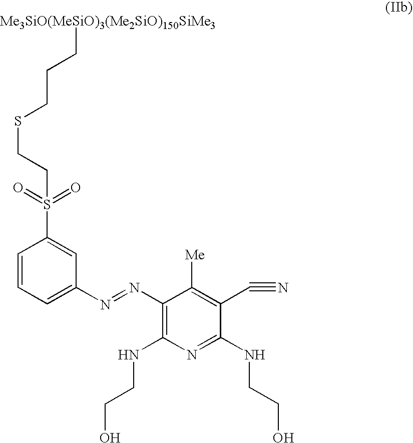

in which Me is methyl;

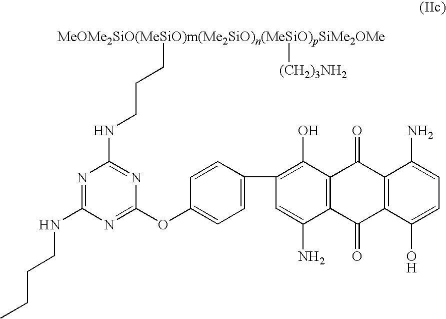

in which Me is methyl and m is 2, n is 100, and p is 1 to 2;

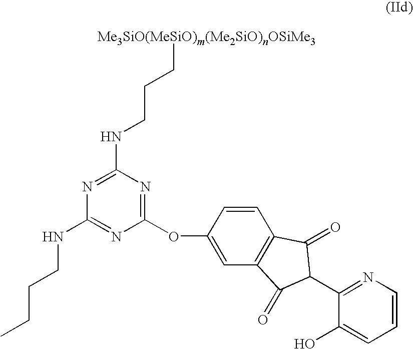

in which Me is methyl and m is 3, and n is 150.

The organopolysiloxanes of the invention have the advantage that apart from the covalently bonded dye radicals they may also have further functional groups, which may endow the compound, additionally to the color, with further properties, such as substantivity, hydrophilicity or hydrophobicity, chemical reactivity, etc., for example. Furthermore, the desired properties of the organopolysiloxanes of the invention can be modified or tailored within a wide range by varying the nature and number of the dye radicals and also of the functional groups and/or their respective proportions.

The organopolysiloxanes of the invention have the advantage, furthermore, that they are stable, in other words that they undergo no substantive alteration for at least one year at room temperature and at the pressure of the surrounding atmosphere, and that they are easily accessible.

The organopolysiloxanes of the invention can be prepared by reacting an organic dye which is free from sulfonic acid groups and sulfonate groups and has a covalently bonded reactive group with an organopolysiloxane which has functional groups which are able to form a covalent bond with the reactive group of the dye.

Particular preference is given to using organic dyes, free from sulfonic acid groups and sulfate groups, that are soluble in silicones. If silicone-soluble reactive dyes are employed there is no need for solvents.

Reactive groups bonded covalently to a dye are, in particular, radicals of the formula —SO₂X¹, where X¹ is halogen, preferably fluoro or chloro;

radicals of the formula —SO₂—(CH₂)₂—V, where V is a moiety which is eliminable by exposure to alkali, and in particular is halogen, preferably chloro, sulfato or thiosulfato;
the radical of the formula —SO₂—CH═CH₂;
radicals of the formula —SO₂—NH—CH₂—CH₂-L, where L is a leaving group, halogen for example, such as fluoro or chloro;
radicals of the formula —NH—CO—CH₂—X², where X² is halogen, such as fluoro or chloro;
radicals of the formula —NH—CO—C(X³)═CH₂ and NH—CO—CH(X³)—CH₂X⁴, where X³ and
X⁴ independently of one another are halogen, such as chloro or bromo; radicals of the formula —NH—CO—CH₂—CH₂R⁴ where R⁴ is halogen, sulfato or sulfinato;
triazine radicals of the formula (III)

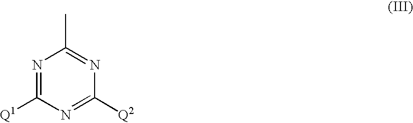

in which
Q¹ and Q² independently of one another are chloro, fluoro, cyanamide, hydroxyl, (C₁-C₆)-alkoxy, phenoxy, sulfophenoxy, mercapto, (C₁-C₆)-alkylmercapto, pyridino, carboxypyridino, carbamoylpyridino, or a group of the formula (IV), (V) or (VI)

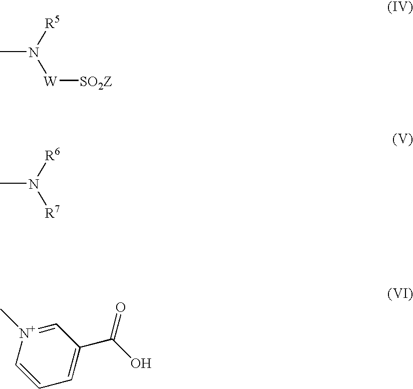

in which
R⁵ is hydrogen, (C₁-C₆)-alkyl, sulfo-(C₁-C₆)-alkyl or phenyl, which is unsubstituted or substituted by (C₁-C₄)-alkyl, (C₁-C₄)-alkoxy, sulfo, halogen, carboxyl, acetamido or ureido;
R⁶ and R⁷ independently of one another have one of the definitions of R⁵ or form a cyclic ring system of the formula —(CH₂)_{q}—, where q is 4 or 5, or of the formula —(CH₂)₂-E-(CH₂)₂—, where E is oxygen, sulfur, sulfonyl or —NR³, and R⁸ is (C₁-C₆)-alkyl;
W is phenylene which is unsubstituted or substituted by one or two substituents, such as (C₁-C₄)-alkyl, (C₁-C₄)-alkoxy, carboxyl, sulfo, chloro, bromo, or (C₁-C₄)-alkylene-arylene or (C₂-C₆)-alkylene which may be interrupted by oxygen, sulfur, sulfonyl, amino, carbonyl, carboxamido, or is phenylene-CONH-phenylene which is unsubstituted or substituted by (C₁-C₄)-alkyl, (C₁-C₄)-alkoxy, hydroxyl, sulfo, carboxyl, amido, ureido or halogen, or is naphthylene which is unsubstituted or substituted by one or two sulfo groups; and

Z is —CH═CH₂ or —SO₂—(CH₂)₂—V, where V is defined as indicated above;

pyrimidine radicals, of formulae (VII) to (X) for example

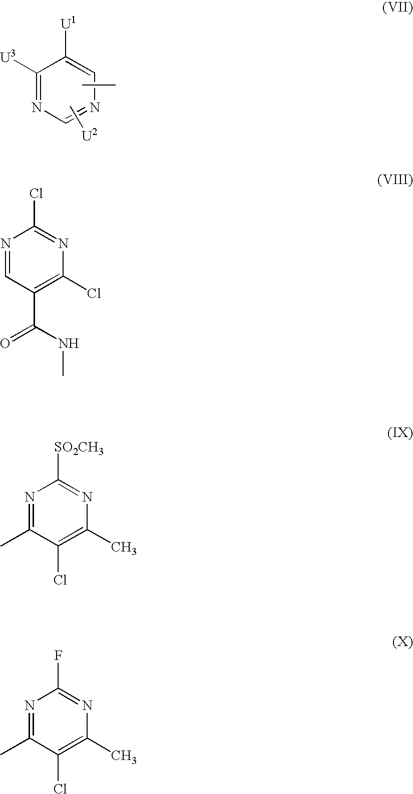

in which
U¹ and U² independently of one another are hydrogen, fluoro or chloro; and
U³ is fluoro or chloro;
quinoxaline radicals, of the formula (XI) for example

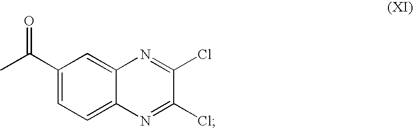

quinazoline radicals, of the formula (XII) for example

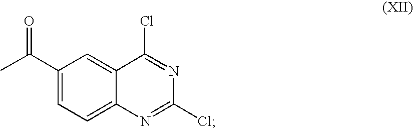

phthalazine radicals, of the formula (XIII) for example

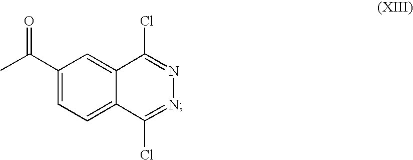

quinoline radicals, of the formula (XIV) for example

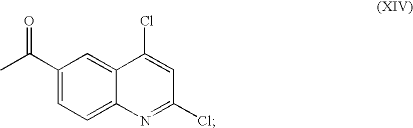

isoquinoline radicals, of the formula (XV) for example

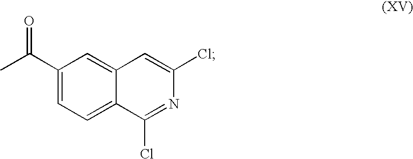

or benzothiazole radicals, of the formula (XVI) for example

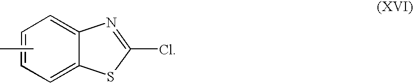

Preferred dyes are those which have radicals of the formula —SO₂X¹, —SO₂—(CH₂)₂—V and triazine radicals of the formula (III) as reactive groups.

The dye is used in amounts of preferably 0.1% to 900% by weight, more preferably 1% to 100% by weight, in particular 5% to 35% by weight, based in each case on the total weight of organopolysiloxane employed. It is advisable in this context to limit the molar amount of dyes to a maximum of 99.9 mol % of the functional groups present in the organopolysiloxane employed.

The dye is used in the process of the invention are known dyes which can either be obtained commercially or be prepared by the methods that are commonplace in organic chemistry and are known to the skilled worker.

Functional groups of the organopolysiloxane which are able to react with reactive groups of the dye are, in particular, amino, mercapto, hydroxyl, carboxyl, acrylate, methacrylate, carbonyl, polyether, and phosphonato, or groups which have glycoside, anhydride, epoxy or silalactone groups or which have quaternary nitrogen. Organopolysiloxanes which have such functional groups and are used in the process of the invention are likewise known products which are available commercially or which are preparable by the methods that are commonplace in silicon chemistry and are known to the skilled worker.

As an example, mention may be made of organosiloxanes which comprise units of the formula (I′)

R¹ₐ(RO)_{b}R′_{c′}R²_{d}SiO_{(4-a-b-c-d/2} (I′),

in which R, R¹, R², a, b, and d are defined as indicated above and R′ can be identical or different and is an amino, mercapto, hydroxyl, carboxyl, anhydride, acrylate, methacrylate, epoxy, quaternary-nitrogen-containing, glycoside-, carbonyl-, polyether-, phosphonato- and/or silalactone-functional hydrocarbon radical, and c′ is as defined for c, with the proviso that the sum of a+b+c′+d is 3, the organopolysiloxanes have at least one radical R′ per molecule, and in the units of the formula (I′) where c′ is other than 0 d is 0.

Examples of R′ are the examples given above for R², preference being given to hydrocarbon radicals substituted by amino groups and derivatives thereof such as the aminomethyl, phenylaminomethyl, aminopropyl, aminoethylaminopropyl, and cyclohexylaminopropyl radical, for example, hydrocarbon radicals substituted by hydroxyl groups, such as primary, secondary or tertiary alcohol radicals, for example, such as the 3-hydroxylpropyl and 4-hydroxybutyl radical, hydrocarbon radicals carrying aromatic hydroxyl groups, such as the phenol or eugenol radical, for example, hydrocarbon radicals substituted by mercapto groups, such as the 3-mercaptopropyl radical, for example, hydrocarbon radicals substituted by carboxylic acid groups or derivatives thereof, such as alkanoic acid radicals, for example, such as the acetyl, 3-carboxypropyl, 4-carboxybutyl, 10-carboxydecyl, and 3-(ethane-1,2-dicarboxyl)propyl radical, acid anhydride radicals, such as the 3-(2,5-dioxotetrahydrofuranyl)propyl radical, and ester radicals, such as the undecene silyl ester radical, and particular preference being given to hydrocarbon radicals substituted by amino groups and derivatives thereof, such as the aminomethyl, aminopropyl, aminoethylaminopropyl and cyclohexylaminopropyl radical, for example, hydrocarbon radicals substituted by hydroxyl groups, such as primary, secondary or tertiary alcohol radicals, for example, such as 3-hydroxypropyl and 4-hydroxybutyl radical, hydrocarbon radicals which carry aromatic hydroxyl groups, such as the phenol or eugenol radical, for example, and hydrocarbon radicals substituted by mercapto groups, such as the 3-mercaptopropyl radical, for example.

The preferred and particularly preferred species of the organopolysiloxanes employed in accordance with the invention are of course structures analogous to those which have already been described above in connection with the organopolysiloxanes of the invention.

The viscosities of the organopolysiloxanes used in accordance with the invention range from preferably 1 mm²/s to 5 000 000 mm²/s, more preferably from 10 mm²/s to 100 000 mm²/s, in each case at 25° C.

The organopolysiloxanes used with particular preference in accordance with the invention are in particular those having an amine number of 0.01 to 10.0, the amine number being the number of mL of a 1 M HCl which are needed to neutralize 1 g of substance.

The process of the invention is preferably carried out largely anhydrously, i.e., in the presence of less than 50 000 ppm of water, preferably less than 10 000 ppm, in particular less than 5000 ppm, based in each case on the total weight of the reaction mixture.

The process of the invention can be carried out in the presence or absence of catalysts. If catalysts are used they may be acidic or basic catalysts. These catalysts may be used either as solids or in the form of their solutions.

Examples of acidic catalysts are Brønsted acids, such as phosphoric acid, sulfuric acid, hydrochloric acid, glacial acetic acid, and formic acid, for example, or Lewis acids, such as lithium perchlorate, zinc tetrafluoroborate, iron(II) chloride, tin(IV) chloride, and Lewis-acidic ionic liquids, for example.

Examples of basic catalysts are primary, secondary or tertiary amines, basic to pyridine, pyrimidine, quinoline, pyridazine, pyrazine, triazine, indole, imidazole, pyrazole, triazole, tetrazole, pyrrole, oxazole, thiazole and/or other N-containing heterocyclic derivatives, basic ammonium salts, such as benzyltrimethylammonium hydroxide and tetramethylammonium hydroxide, alkali metal hydroxides, alkaline earth metal hydroxides, alkali metal alkoxides, alkali metal amides, and Lewis-basic is ionic liquids.

If a catalyst is used in the reaction of the invention the amounts involved are from preferably 0.1% to 1% by weight, based on the total weight of the reactants.

The process of the invention can be carried out with or without solvent as a single-phase or multiphase reaction, in dispersion (solid-liquid or liquid-liquid, such as microemulsions or macroemulsions, for example), preference being given to its being carried out in dispersion, including in aqueous dispersions, with the objective of the generation of very small dye particle sizes of between 1 and 10 μm, and particular preference therefore being given to its being carried out as a homogeneous single-phase reaction.

If solvent is used for the process of the invention, the solvents in question are preferably inert solvents without effect on the course of the reaction. Examples of suitable solvents, which for the process of the invention can be employed individually or in a mixture with one another, are pentane, petroleum ether, n-hexane, hexane isomixtures, cyclohexane, heptane, octane, wash benzine, decalin, benzene, toluene, xylene, diethyl ether, di-n-propyl ether, diisopropyl ether, di-n-butyl ether, methyl tert-butyl ether, ethylene glycol monomethyl ether, ethylene glycol monobutyl ether, ethylene glycol dimethyl ether, ethylene glycol diethyl ether, diglycol, diethylene glycol monomethyl ether, diethylene glycol monoethyl ether diethylene glycol monobutyl ether, diethylene glycol dimethyl ether, diethylene glycol diethyl ether, diethylene glycol dibutyl ether, methanol, ethanol, isopropanol, butanol, pentanols, n-hexanol, 1,2-propanediol, 1,3-propanediol, ethylene glycol, glycerol, tetrahydrofuran, dioxane, methyl acetate, ethyl acetate, n-, sec-, and tert-butyl acetate, dichloromethane, trichloromethane, tetrachloromethane, 1,2-dichloroethane, trichloroethylene, tetrachloroethylene, chlorobenzene, 1-chloronaphthalene, ethylene carbonate, propylene carbonate, CO₂, acetonitrile, acetamide, tetrahydro-1,3-dimethyl-2(1H)-pyrimidinone (DMPU), hexamethylphosphoric triamide (HMPT), dimethyl sulfoxide (DMSO), sulfolane, acetone, methyl ethyl ketone (MEK), methyl isobutyl ketone (MIBK), diisopropyl ketone, ionic liquids, linear and cyclic siloxanes, and mixtures of said solvents.

If the process of the invention is carried out as a two-phase reaction (liquid-liquid), which is not preferred, however, then it is necessary to ensure maximum homogenization of the mutually immiscible phases and the creation of a large internal reaction surface area, as for example by generating an average particle size of <500 μm. The intensive commixing of the reaction phases may be accomplished in principle by any of the known, prior-art mixing systems, such as, for example, stirrers of all kinds, high-speed stirrers and high-performance dispersers, such as those available under the brand name IKA Ultra-Turrax® or a similar dissolver system, by means of ultrasound probes or ultrasonic baths, electrical, magnetic or electromagnetic fields, etc., or—as for example in the case of the continuous reaction regime—with static or moving mixing elements or mixing nozzles, and also by turbulent flow, or by any desired combinations thereof.

Where the process of the invention is carried out in dispersion, it is possible accordingly for dispersing assistants (e.g., emulsifiers) or interface-active or surface-active agents to be present, such as nonionic, anionic, cationic or amphoteric emulsifiers, it being possible for the preparation of the dispersion to take place in any desired manner known to the skilled worker.

The reactants used in the process of the invention may in each case be one kind of such a component or else a mixture of at least two kinds of a respective component. In the process of the invention the reactants used can be mixed with one another, supplied to the reaction and/or brought to reaction in any desired way known per se. The process of the invention can be carried out batchwise, semibatchwise or continuously in reactor systems suitable for the purpose, such as batch reactors, batch reactor cascades, loop reactors, flow tubes, tube reactors, microreactors, circulation pumps, for example, and in any desired combinations thereof.

After the end of the reaction of the invention, the reaction products can be separated from any reaction auxiliaries employed, and isolated, by any desired process steps which are known per se. Examples are filtration, centrifugation, and extraction. If desired, volatile components and any solvent used can also be removed, by distillation, after the reaction.

The process of the invention may additionally be followed by any desired further is operating steps, by means of which it is possible to tailor the desired properties of the organopolysiloxanes of the invention. By this means it is possible, for example, to set with precision the desired molecular weight, a specific distribution of the dye radicals in the molecule, and, if desired, the introduction of further functionalities. The implementation of the operating steps is oriented in this case fundamentally on the present state of the art, and takes place in a way which is known to the skilled worker.

Examples of such subsequent reactions are, in particular, equilibration reactions with, for example, organopolysiloxanes, condensation with other organosilicon compounds capable of condensation reactions, such as silanols, alkoxy- or chloro-functional silanes, and silanol-, alkoxy- or chloro-functional polysiloxanes or silicone resins, silica or highly disperse silicic acid (e.g., WACKER HDK®), for example, and also the further organofunctional modification of the organosilicon compound.

The process of the invention boasts a range of advantages over the prior art. It is preparatively simple and can be realized without special apparatus. Through the use of reactive dyes free from sulfonic acid groups and sulfonate groups, which are oleophilic and highly silicone-compatible, the reaction of the invention can be configured as a homogeneous single-phase reaction without the addition of sizeable amounts of compatibilizing solvents, as are required, for instance, for a homogeneous reaction regime in the case of the water-soluble reactive dyes mentioned in WO 98/40429 A1, which contain sulfonic acid groups and/or sulfonate groups; after the reaction, such solvents have to be removed again, which is costly and inconvenient. The single-phase reaction not only allows outstanding reaction yields in short reaction times but also makes the process of the invention cost-effective, sparing in its use of resources, and, moreover, sustainedly environment-compatible.

The process of the invention is suited equally to a discontinuous regime as to a continuous regime, implying a further advantage in respect of costs, flexibility, and space-time yield.

The organopolysiloxanes of the invention can be used as colorants. The substrates for coloring in this case encompass a multiplicity of materials, described in more detail below.

The organopolysiloxanes of the invention can be used with particular advantage in those instances where value is placed on the combination of properties typical for silicones, such as water repellency, dirt repellency, protection, soft hand, gloss, etc., with a visible or latent coloration.

In the cosmetic applications field, suitable applications include in particular those in decorative cosmetology, skincare, and haircare. Typical haircare applications are for example the permanent, semipermanent or temporary coloring of keratinic fibers by cosmetic formulations which comprise the organopolysiloxanes of the invention as coloring ingredients. Further benefits which may be obtained, besides the coloring or shading, include, for example, the heightening of the hair's gloss, of its volume, and of its curl retention, an improved softness to the touch, an improvement in dry or wet combability through a reduction in the combing resistance, a reduction in the antistatic charging, and the general protection of the keratinic fiber against splitting, becoming dry, and structurally harmful environmental effects.

In the skincare sector as well it is possible to use the organopolysiloxanes of the invention—for example, as a lipophilic formulating ingredient in makeup, lipstick, lipgloss, mascara, eyeliner, nail varnish, massage oil or massage gel, in skin creams or in sun care products. Benefits typical of silicones include in this context, for example, a pleasant skin sensation, a general reduction in the stickiness of the cosmetic formulation, a reduction in the propensity of any pigments or fillers present to undergo aggregation, and also the development of a hydrophobic but breathable barrier on the skin surface, which leads, for example, to improved water resistance on the part of the cosmetic product.

In addition it is possible to color cosmetics or household products with the organopolysiloxanes of the invention in order to draw particular attention to active components or—for marketing reasons, for example—to carry out optical upgrading of products (increasing the product's attractiveness).

The organopolysiloxanes of the invention are also outstandingly suitable, furthermore, for paper, tissue, leather, and textile applications. The treatment of these substrates may on the one hand be carried out only for purely decorative or fashion reasons or may serve a substrate care purpose, as for example when the color of colored textiles is re-established or re-emphasized by means of recoloring products. On the other hand, as well as imparting color, it is possible to obtain a series of positive benefits which are otherwise achievable only by means of multistage treatment methods. By way of example, paper towels, textiles, yarns, woven fabrics, natural or synthetic fibers can in one operation be colored and at the same time be provided with the desired hand properties (soft, flowing, velvety, smooth or the like). In the same way the coloring operation can also be combined with substrate hydrophilization or, in particular, with substrate hydrophobization. In contrast to hydrophilic finishes in the tissue and textile sector, mention may be made here, by way of example, of the treatment of leather, where in the wet-end process, for example, the colored organosilicon compounds can be used to obtain full and uniform deep-down coloring in conjunction with water repellency. In the fabric care sector, conversely, the hydrophilization and softening of textiles are desired, in combination with a deepening of color, regeneration of color or optical brightening in the course of the laundering operation.

The organopolysiloxanes of the invention can also be used, furthermore, in abhesive, reprographic, and printing applications. In the case of release papers siliconized differently on either side or siliconized on one side, for example, it is useful to be able to distinguish the sides visually by means of colored marking. The organopolysiloxanes of the invention are especially suitable for this purpose, since unlike conventional organic dyes they do not affect the abhesive properties of the release papers. Moreover, the organopolysiloxanes of the invention can be used as an ingredient of toners or in formulations for color printing. When employed as a color assist additive in textile pigment printing, the organopolysiloxanes of the invention lead to a range of desired benefits, such as deepening of color, greater brilliance of color, provision of gloss, or improved rub fastness properties, for example.

Conventional architectural preservation and textile construction are two further fields of application for the organopolysiloxanes of the invention. Both in architectural preservation (maintaining built structures, ensuring the long-term stability of buildings, and imparting water repellency to building materials) and in textile construction, silicon-based products play an important part. In the context of the color modification of such products, the requirement is not only for 100% compatibility between the components, the majority of which are silicon-based, but also for assistance in respect of water repellency, water vapor permeability, and long-term resistance of the coating toward environmental effects. All of these requirements are met by the organopolysiloxanes of the invention, which are therefore outstandingly suitable for use as a colored formulating ingredient of architectural preservation coatings, wall paints or varnishes, for the coloring of mass-hydrophobized or surface-hydrophobized mineral building materials, and also for the color modification of textile coatings and siliconized textile wovens, knits or form-loop products, of the kind used, for example, for window panels, conveyor belts, safety clothing or protective clothing.

The organopolysiloxanes of the invention are suitable, furthermore, for polish applications, with very different effects being obtainable depending on the nature of the substrate and the thickness of the applied layer. For example, the organopolysiloxanes of the invention can be used in paint care (in the automobile sector, for example), in polishes for leather, furniture or lacquered articles, and also in hard wax care products, where typical target effects include color intensification, color regeneration, color shading, and the masking of irregularities or scratches. In the shoe polish sector the organopolysiloxanes of the invention contribute to hydrophobizing the outer leather, deepening color, and boosting shine.

Furthermore, the organopolysiloxanes of the invention are extremely suitable for coloring polymers, polymer blends, polymer compounds, or any of a very wide variety of plastics which can be produced from them. In particular they are suitable for coloring thermoplastics, such as polyethylene, polypropylene, polystyrene, polyamides, polyesters, polycarbonates, polyoxymethylene, polyvinyl chloride or acrylonitrile-butadiene-styrene copolymers.

The organopolysiloxanes of the invention are suitable, moreover, in particular for the coloring of silicon polymers of all kinds, such as silicones and silicone elastomers, resins, and waxes, for example, the organopolysiloxanes of the invention being distributed homogeneously in the polymer as molecular, coloring constituents and as such being no longer extractable from the polymer. In this context the advantage of the colored organopolysiloxanes of the invention in carrying not only the coloring groups but also further functional groups on the silicone backbone becomes clear, since these further functional groups can be selected such that vulcanization with the silicon polymers of all kinds is achieved, resulting in maximum transparency and compatibility and also preventing the migration of the coloring components. In addition, the high transparency of the organopolysiloxanes of the invention makes it possible to obtain very clear transparent coloring of polymers in conjunction with high translucency over a broad spectral range.

In addition to the applications mentioned so far, the organopolysiloxanes of the invention are also suitable as marker substances for the investigation of processes of migration, penetration, sedimentation or coating, as for example in the context of the determination of penetration depths, of applied layer thicknesses, weights, and homogeneities, in the monitoring of flows of product or compound, and in the investigation of the processes underlying a finishing operation (such as the finishing of natural or synthetic fibers with silicone products, for example). If the dye radicals of the organopolysiloxanes of the invention are UV-active, fluorescent, phosphorescent, or enzymatically, chemically or physically stimulatable chromophores, the organopolysiloxanes of the invention can also be used as a hidden company seal for the discreet marking of products or formulations.

In general the organopolysiloxanes of the invention are also suitable for obtaining a visual indication of the homogeneity of a product or a product formula or of its correct application. The latter is highly important in particular in areas where it is necessary for one or more products to be applied to or distributed on an area as uniformly as possible, as in the case, for example, of abhesive paper coatings, of sunscreens or similar sun care products, of pharmaceutical products, and of medical products (in cases of extensive topical application, for example).

The organopolysiloxanes of the invention are also suitable, finally, for tinting lipophilic substrates in the food, agricultural, and pharmaceutical sectors.

The examples below illustrate the invention. All parts given with percentages refer to the weight unless otherwise indicated. Moreover, all viscosity figures relate to a temperature of 25° C. Unless indicated otherwise, the examples below are carried out under the pressure of the surrounding atmosphere, in other words at approximately 1000 hPa, and at room temperature, in other words at about 20° C., or at a temperature which comes about when the reactants are combined at room temperature without additional heating or cooling.

### EXAMPLE 1

440 mg (991 μmol) of dye having the following structure

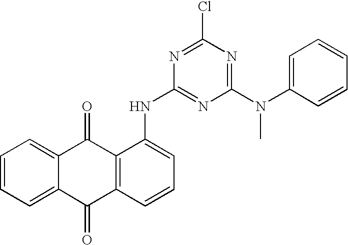

were metered into 110 g of an aminoalkyl-carrying polydimethylsiloxane (amine number: 90 μmol of amino groups per gram; viscosity: 601 mm²/s) and distributed homogeneously in the siloxane for 10 minutes using a high-performance disperser (e.g., IKA Ultra-Turrax®). The mixture was then heated with stirring at 100° C. for 4 h. After cooling to room temperature, the product was filtered through a depth filter. This gave 100 g (91%) of a yellow-colored silicone oil.

### EXAMPLE 2

440 mg (991 μmol) of the dye described in example 1 were metered into 110 g of an aminoalkyl-carrying polydimethylsiloxane (amine number: 90 μmol of amino groups per gram; viscosity: 601 mm²/s) and distributed homogeneously in the siloxane for 10 minutes using a high-performance disperser (e.g., IKA Ultra-Turrax®). Then 700 mL of toluene were added with stirring and the mixture was heated at reflux temperature for 4 h with stirring. After cooling to room temperature, the product was filtered through a depth filter. This gave 99 g (90%) of a yellow-colored silicone oil.

### EXAMPLE 3

3.52 g of a dispersion of the dye described in example 1 (991 mmol) in water (12.5% by weight; prepared by grinding of the colorant in a bead mill) were metered into 110 g of an aminoalkyl-carrying polydimethylsiloxane (amine number: 90 μmol of amino groups per gram; viscosity: 601 mm²/s). The mixture was subsequently heated at 100° C. for 4 h with stirring. After it had cooled to room temperature, all of the volatile components were removed in vacuo and the residue obtained was filtered through a depth filter in order to remove unreacted constituents. This gave 99 g (90%) of a yellow-colored silicone oil.

### EXAMPLE 4

540 mg (984 μmol) of dye having the following structure

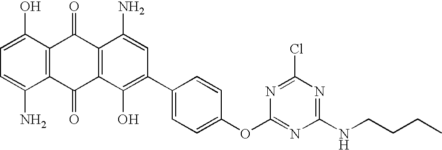

were metered into 110 g of an aminoalkyl-carrying polydimethylsiloxane (amine number: 90 μmol of amino groups per gram; viscosity: 601 mm²/s) and distributed homogeneously in the siloxane for 10 minutes using a high-performance disperser (e.g., IKA Ultra-Turrax®). The mixture was then heated with stirring at 100° C. for 4 h. After cooling to room temperature, the product was filtered through a depth filter. This gave 100 g (91%) of a blue-colored silicone oil.

### EXAMPLE 5

560 mg (991 μmol) of dye having the following structure

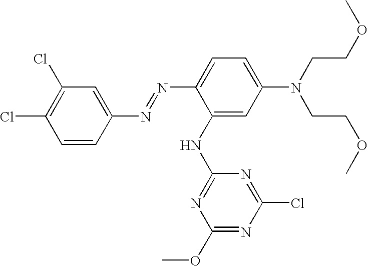

were metered into 110 g of an aminoalkyl-carrying polydimethylsiloxane (amine number: 90 μmol of amino groups per gram; viscosity: 601 mm²/s) and distributed homogeneously in the siloxane for 10 minutes using a high-performance disperser (e.g., IKA Ultra-Turrax®). The mixture was then heated with stirring at 100° C. for 4 h. After cooling to room temperature, the product was filtered through a depth filter. This gave 98 g (89%) of an orange-colored silicone oil.

### EXAMPLE 6

560 mg (995 μmol) of dye having the following structure

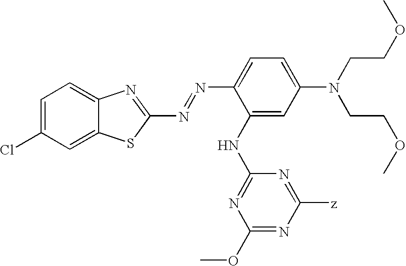

were metered into 110 g of an aminoalkyl-carrying polydimethylsiloxane (amine number: 90 μmol of amino groups per gram; viscosity: 601 mm²/s) and distributed homogeneously in the siloxane for 10 minutes using a high-performance disperser (e.g., IKA Ultra-Turrax®). The mixture was then heated with stirring at 100° C. for 4 h. After cooling to room temperature, the product was filtered through a depth filter. This gave 99 g (90%) of a ruby-colored silicone oil.

### EXAMPLE 7

490 mg (996 μmol) of dye having the following structure

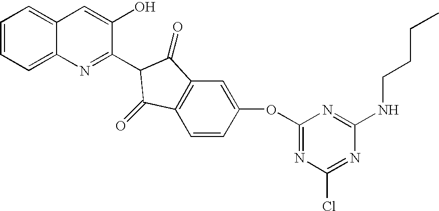

were metered into 110 g of an aminoalkyl-carrying polydimethylsiloxane (amine number: 90 μmol of amino groups per gram; viscosity: 601 mm²/s) and distributed homogeneously in the siloxane for 10 minutes using a high-performance disperser (e.g., IKA Ultra-Turrax®). The mixture was then heated with stirring at 100° C. for 4 h. After cooling to room temperature, the product was filtered through a depth filter. This gave 97 g (88%) of a yellow-colored silicone oil.

### EXAMPLE 8

7.3 g of a dispersion of the dye described in example 7 (996 μmol) in water (6.7% by weight; prepared by grinding of the colorant in a bead mill) were metered into 110 g of an aminoalkyl-carrying polydimethylsiloxane (amine number: 90 μmol of amino groups per gram; viscosity: 601 mm²/s). The mixture was subsequently heated at 100° C. for 4 h with stirring. After it had cooled to room temperature, all of the volatile components were removed in vacuo and the residue obtained was filtered through a depth filter in order to remove unreacted constituents. This gave 100 g (91%) of a yellow-colored silicone oil.

### EXAMPLE 9

530 mg (993 μmol) of dye having the following structure

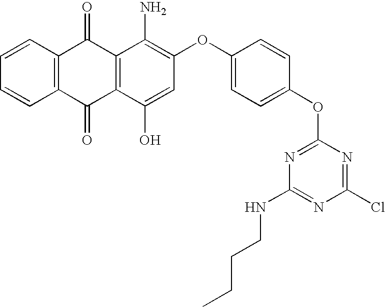

were metered into 110 g of an aminoalkyl-carrying polydimethylsiloxane (amine number: 90 μmol of amino groups per gram; viscosity: 601 mm²/s) and distributed homogeneously in the siloxane for 10 minutes using a high-performance disperser (e.g., IKA Ultra-Turrax®). The mixture was then heated with stirring at 100° C. for 4 h. After cooling to room temperature, the product was filtered through a depth filter. This gave 98 g (89%) of a red-colored silicone oil.

In the same way as in the above examples it is also possible to obtain inventive silicone oils with the dye radicals (A19) to (A38).

1-9. (canceled)

10. A colored organopolysiloxane comprising units of the formula
R¹ₐ(RO)_{b}A_{c}R²_{d}SiO_{(4-a-b-c-d)/2} (I),
in which
R can be identical or different and is hydrogen or a monovalent, unsubstituted or substituted hydrocarbon radical;
R¹ can be identical or different and is hydrogen or a monovalent, SiC-bonded, unsubstituted or substituted hydrocarbon radical;
R² can be identical or different and is a substituted monovalent hydrocarbon radical;
A can be identical or different and is an organic dye radical free from sulfonic acid groups and sulfonate groups;
a is 0, 1, 2 or 3;
b is 0, 1, 2 or 3;
d is 0, 1, 2 or 3;
and c is 0, 1 or 2;
with the proviso that the sum a+b+c+d is ≦3, the organopolysiloxane has at least one radical A per molecule, and in the units of the formula (I) where c is other than 0 when d is 0.

11. The colored organopolysiloxane as claimed in claim 10, wherein R is hydrogen, methyl, ethyl or propyl.

12. The colored organopolysiloxane as claimed in claim 10, wherein R¹ is methyl, vinyl or allyl.

13. The colored organopolysiloxane as claimed in claim 10, wherein R² is aminopropyl, aminoethylaminopropyl, hydroxypropyl or mercaptopropyl.

14. The colored organopolysiloxane as claimed in claim 10, which is of the formula (II)
AₘR²ₙR¹₃₋ₘ₋ₙSiO-(A₂SiO)ₑ—(R¹_{f}R²_{2-f}SiO)_{g}—(R²ₕR¹₂₋ₕSiO)₁—(R¹ⱼAR²₁₋ⱼSiO)ₖ—SiAₘR²ₙR¹3-m-n (II)
in which
R¹, R² and A are defined as in claim 10;
f is 0 or 1;
h is 0, 1 or 2;
j is 0 or 1;
m is 0 or 1;
n is 0 or 1;
e is 0 or an integer from 1 to 100;
g is 0 or an integer from 1 to 100;
i is 0 or an integer from 1 to 100;
k is an integer from 1 to 100;
it being possible for the subunits in the formula (II) to be distributed randomly in the molecule.

15. The colored organopolysiloxane as claimed in claim 14, wherein
f is 1;
h is 0; and
j is 1.

16. The colored organopolysiloxane as claimed in claim 10, wherein the dye content is 0.1% to 90% by weight, based in each case on the total weight of the colored organopolysiloxane.

17. The colored organopolysiloxane as claimed in claim 10, wherein the dye content is 0.5% to 50% by weight, based in each case on the total weight of the colored organopolysiloxane.

18. The colored organopolysiloxane as claimed in claim 10, wherein the dye content is 5% to 25% by weight, based in each case on the total weight of the colored organopolysiloxane.

19. A process for preparing a colored organopolysiloxane as claimed in claim 10, which comprises reacting an organic dye which is free from sulfonic acid groups and sulfonate groups and has a covalently bonded reactive group with an organopolysiloxane that has functional groups which are able to form a covalent bond with the reactive group of the dye.

20. The process as claimed in claim 19, wherein the organic dye used that is free from sulfonic acid groups and sulfonate groups is soluble in silicones.

21. A colorant which comprises the colored organopolysiloxane as claimed in claim 10.
